(19) United States
(12) Patent Application Publication
(10) Pub. No.: US 2023/0200393 A1
(43) Pub. Date: Jun. 29, 2023

(54) SELECTIVE HERBICIDES BASED ON SUBSTITUTED ISOXAZOLIN CARBOXAMIDES AND METCAMIFEN

(71) Applicant: Bayer Aktiengesellschaft, Leverkusen (DE)

(72) Inventors: Jan DITTGEN, Frankfurt (DE); Elmar GATZWEILER, Bad Nauheim (DE); Christopher Hugh ROSINGER, Hofheim (DE); Lothar LORENTZ, Waldbroel (DE); Klaus Bernhard HAAF, Kelkheim (DE); Klaus TRABOLD, Heidelberg (DE); Hubert MENNE, Mainz-Kastel (DE); Julio PEREZ CATALAN, Duesseldorf (DE)

(21) Appl. No.: 18/000,361

(22) PCT Filed: May 31, 2021
(86) PCT No.: PCT/EP2021/064498

§ 371 Date: Nov. 30, 2022

(30) Foreign Application Priority Data
Jun. 2, 2020 (EP) 20177909.7

(51) Int. Cl. A01N 43/80; A01N 47/30; A01P 13/00

(52) U.S. Cl. CPC A01N 43/80; A01N 47/30; A01P 13/00

(57) ABSTRACT

The invention relates to novel selective herbicidal active compound combinations which comprise substituted isoxazolincarboxamides or agrochemical acceptable salts thereof and metcamifen and which can be used with particularly good results for the selective control of weeds in various crops of useful plants.

The invention relates to novel selective herbicidal active compound combinations which comprise substituted isoxazolincarboxamides or agrochemical acceptable salts thereof and metcamifen and which can be used with particularly good results for the selective control of weeds in various crops of useful plants.

Substituted isoxazolincarboxamides are already known as effective herbicides from WO 2018/228985 and WO 2019/145245. However, the activity of these compounds and/or their compatibility with crop plants are not entirely satisfactory under all conditions.

Metcamifen (IUPAC: 2-methoxy-N-({4-[(methylcarbamoyl)amino]phenyl}sulfonyl)benz-amide; CAS: 129531-12-0) is a safener known from EP 0365484.

Surprisingly, it has now been found that certain substituted isoxazolincarboxamides, when used together with metcamifen described below, prevent damage to crop plants extremely well and can be used particularly advantageously as broad-spectrum combination preparations for the selective control of weeds (=undesirable plants) in crops of useful plants, such as, for example, in cereals and maize.

The invention provides selective herbicidal combinations comprising
(a) substituted isoxazolincarboxamides of the formula (I) or agrochemical acceptable salts thereof

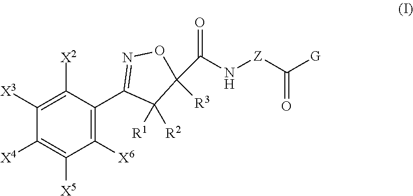

in which
G represents OR⁴ or NR⁷R⁸
R¹ and R² each represent hydrogen;
R³ represents (C₁-C₅)-alkyl, (C₃-C₆)-cycloalkyl, (C₂-C₅)-alkenyl, (C₂-C₅)-alkinyl or (C₁-C₅)-alkoxy each optionally substituted “m” times by substituents from the group consisting of halogen, cyano, (C₁-C₅)-alkoxy and hydroxy;
R⁴ represents hydrogen,
   or
   represents (C₁-C₁₂)-alkyl, (C₃-C₇)-cycloalkyl, (C₃-C₇)-cycloalkyl-(C₁-C₅)-alkyl, (C₂-C₅)-alkenyl, (C₅-C₆)-cycloalkenyl, (C₁-C₄)-alkylphenyl or (C₂-C₅)-alkinyl each optionally substituted “m” times by substituents from the group consisting of halogen, cyano, (C₁-C₆)-alkoxy, (C₁-C₆)alkoxycarbonyl, hydroxy, S(O)ₙR⁵;
R⁵ represents (C₁-C₅)-alkyl, (C₂-C₅)-alkenyl, (C₃-C₆)-cycloalkyl, benzyl, CON((C₁-C₃)-alkyl)₂ or (C₁-C₅)-alkyl-C(O)—(C₁-C₅)-alkyl each optionally substituted “m” times by substituents from the group consisting of halogen and cyano;
R⁶ represents hydrogen,
   or
   represents (C₁-C₈)-alkyl, (C₃-C₆)-cycloalkyl, (C₃-C₈)-alkenyl or (C₃-C₈)-alkinyl each optionally substituted “m” times by substituents from the group consisting of halogen, cyano und (C₁-C₂)alkoxy;
R⁷, R⁸ independently of each other represent hydrogen, (C₁-C₆)-alkoxycarbonyl-(C₁-C₆)-alkyl, N((C₁-C₃)-alkyl)₂, S(O)ₙR⁵,
   or
R⁷ and R⁸ together with the nitrogen atom to which they are attached form a saturated or partially or fully unsaturated five-, six-, or seven-membered ring which may contain apart from the nitrogen atom “r” carbon atoms, “o” oxygen atoms and is optionally substituted “m” times by substituents from the group consisting of halogen, (C₁-C₆)-alkyl, halogen-(C₁-C₆)-alkyl, oxo, CO₂R⁶;
Z represents Z-1 to Z-8:

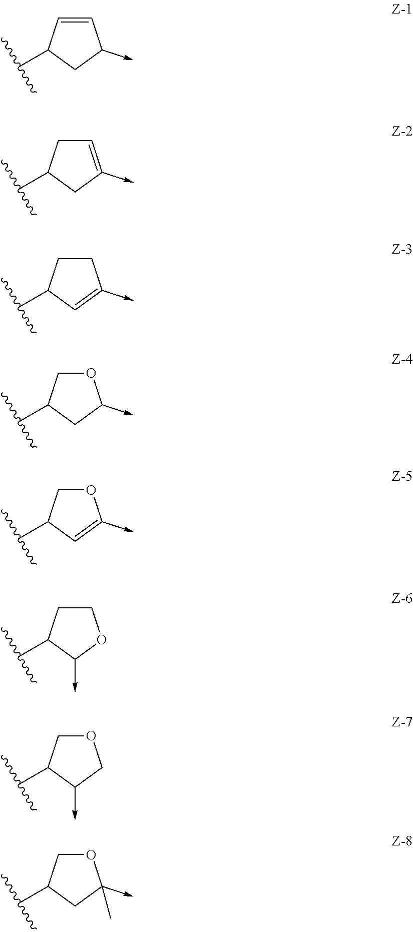

whereas the arrow represents the bonding to the group CO-G of the formula (I);
X², X⁴ and X⁶ independently of one another represent hydrogen or fluorine;
X³ and X⁵ independently of one another represent hydrogen, chlorine, cyano or fluorine;
   or
   represents (C₁-C₃)-Alkyl, (C₁-C₃)-Alkoxy each optionally substituted “m” times by substituents from the group consisting of fluorine or chlorine;
m represents 0, 1, 2, 3, 4 or 5;
n represents 0, 1 or 2;
o represents 0, 1 or 2;
r represents 3, 4, 5 or 6;
and
(b) metcamifen.

### Definitions

Halogen represents radicals of fluorine, chlorine, bromine and iodine. Preference is given to the radicals of fluorine and chlorine.

Alkyl means saturated straight-chain or branched hydrocarbyl radicals having the number of carbon atoms specified in each case, e.g. C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Alkenyl means unsaturated straight-chain or branched hydrocarbyl radicals having the number of carbon atoms specified in each case and one double bond in any position, e.g. C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkynl represents a straight-chain or branched hydrocarbyl groups having 2 to 8, preferably 2 to 6, carbon atoms and one triple bond in any position. Non-limiting examples include ethynyl, prop-1-ynyl, prop-2-ynyl, but-1-ynyl, but-2-ynyl, but-3-ynyl, 1-methylprop-2-ynyl, pent-1-ynyl, pent-2-ynyl, pent-3-ynyl, pent-4-ynyl, 1-methylbut-2-ynyl, 1-methylbut-3-ynyl, 2-methylbut-3-ynyl, 3-methylbut-1-ynyl, 1,1-dimethylprop-2-ynyl, 1-ethylprop-2-ynyl, hex-1-ynyl, hex-2-ynyl, hex-3-ynyl, hex-4-ynyl, hex-5-ynyl, 1-methylpent-2-ynyl, 1-methylpent-3-ynyl, 1-methylpent-4-ynyl, 2-methylpent-3-ynyl, 2-methylpent-4-ynyl, 3-methylpent-1-ynyl, 3-methylpent-4-ynyl, 4-methylpent-1-ynyl, 4-methylpent-2-ynyl, 1,1-dimethylbut-2-ynyl, 1,1-dimethylbut-3-ynyl, 1,2-dimethylbut-3-ynyl, 2,2-dimethylbut-3-ynyl, 3,3-dimethylbut-1-ynyl, 1-ethylbut-2-ynyl, 1-ethylbut-3-ynyl, 2-ethylbut-3-ynyl and 1-ethyl-1-methylprop-2-ynyl.

Cycloalkyl means a carbocyclic saturated ring system having preferably 3-8 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene.

Alkoxy means saturated straight-chain or branched alkoxy radicals having the number of carbon atoms specified in each case, for example C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Halogen-substituted alkoxy means straight-chain or branched alkoxy radicals having the number of carbon atoms specified in each case, where some or all of the hydrogen atoms in these groups may be replaced by halogen atoms as specified above, e.g. C₁-C₂-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-1,2-difluoroethoxy, 2,2-dichloro-2-fluoroethoxy, 2,2,2-trichloroethoxy, pentafluoroethoxy and 1,1,1-trifluoroprop-2-oxy.

The compounds according to the invention are defined in general terms by the formula (I). Preferred substituents or ranges of the radicals given in the formulae mentioned above and below are illustrated hereinafter:

Preferred are herbicidal combinations wherein compound of formula (I) is one of the compounds of the formula (Ia) or agrochemical acceptable salts thereof

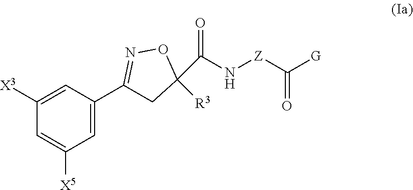

in which
X³, X⁵, R³ and G are as described above;
Z means Z-1a, Z-1b, Z-2a, Z-3a, Z-4a, Z-5a, Z-6a, Z-7a, Z-8a,

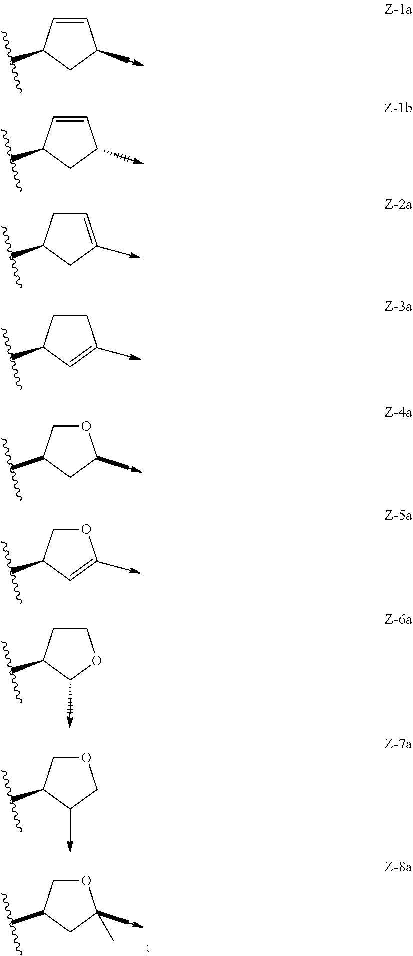

wherein Z-4a means the mixture of both structures Z-4b and Z-4c;

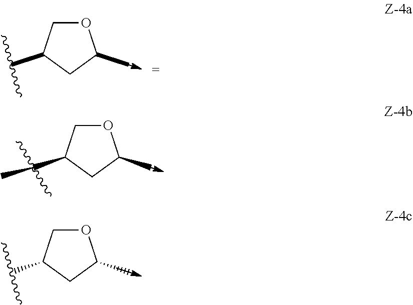

and wherein Z-8a means the mixture of both structures Z-8b and Z-8c

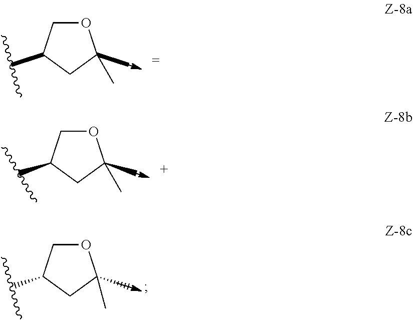

and wherein the arrow means a bond to the group CO-G in formula (Ia).

Especially preferred are herbicidal combinations wherein compound of formula (Ia) is one of the compounds of table 1 or agrochemical acceptable salts thereof.

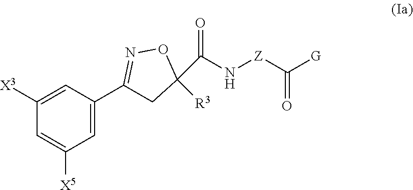

TABLE 1
Examples for compounds of the formula (Ia)

| No. | X³ | X⁵ | R³ | 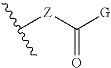 |
|---|---|---|---|---|
| 1.1 | F | F | (R)—CH₂Cl | 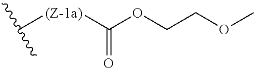 |
| 1.2 | F | CN | (R)—CF₃ | 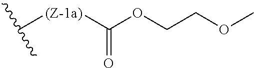 |
| 1.3 | F | F | (R)—CF₃ | 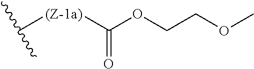 |
| 1.4 | F | H | (R)—CH₃ | 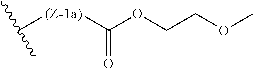 |
| 1.5 | Cl | CN | (R)—CH₃ | 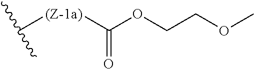 |
| 1.6 | F | H | (S)-vinyl | 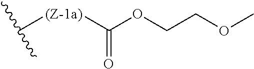 |
| 1.7 | F | F | (R)—CF₃ | 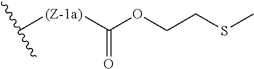 |
| 1.8 | F | CH₃ | (R)—CF₃ | 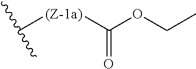 |
| 1.9 | H | H | (R)—CF₃ | 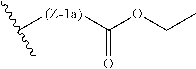 |
| 1.10 | F | F | (R)—CH₃ | 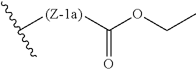 |
| 1.11 | H | H | (R,S)—CF₃ | 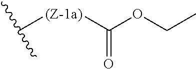 |
| 1.12 | OCH₃ | OCH₃ | (R,S)—CF₃ | 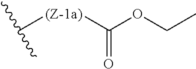 |
| 1.13 | F | F | (R,S)—CH₃ | 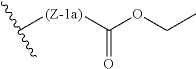 |
| 1.14 | F | F | (S)-vinyl | 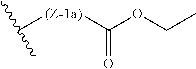 |
| 1.15 | F | F | (R,S)—OCH₃ | 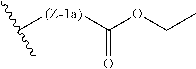 |
| 1.16 | F | F | (R)—OCH₃ | 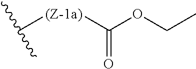 |
| 1.17 | F | F | (R)—CH₂F | 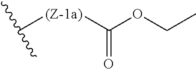 |
| 1.18 | F | H | (R)—CF₃ | 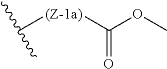 |
| 1.19 | F | F | (R)—CH₃ | 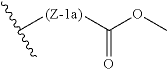 |
| 1.20 | F | H | (R)—CH₃ | 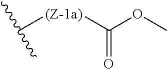 |
| 1.21 | F | Cl | (R)—CH₃ | 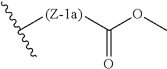 |
| 1.22 | F | CH₃ | (R,S)—CF₃ | 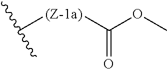 |
| 1.23 | Cl | CN | (R,S)—CH₃ | 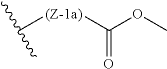 |
| 1.24 | F | CN | (R,S)—CH₃ | 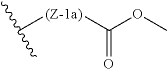 |
| 1.25 | F | H | (S)-vinyl | 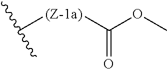 |
| 1.26 | F | F | (S)-vinyl | 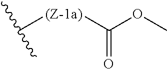 |
| 1.27 | F | F | (R)—CH₃ | 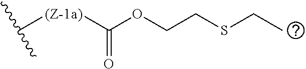 |
| 1.28 | F | H | (R)—CH₃ | 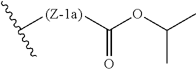 |
| 1.29 | F | F | (R)—CH₃ | 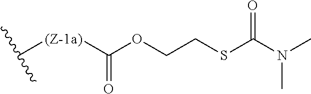 |
| 1.30 | F | F | (R)—CH₃ | 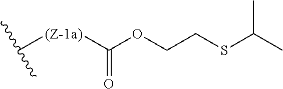 |
| 1.31 | F | F | (R)—CH₃ | 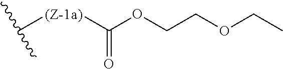 |
| 1.32 | F | H | (R)—CH₃ | 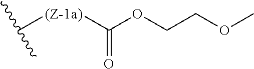 |
| 1.33 | F | F | (R)—CH₃ | 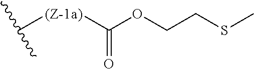 |
| 1.34 | F | F | (R)—CH₃ | 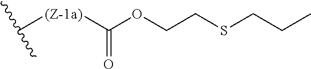 |
| 1.35 | F | F | (R)—CH₃ | 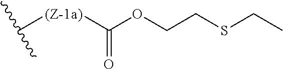 |
| 1.36 | F | F | (R)—CH₃ | 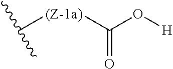 |
| 1.37 | F | F | (R)—CH₃ | 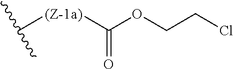 |
| 1.38 | F | F | (R)—CH₃ | 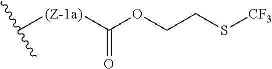 |
| 1.39 | F | H | (R)—CH₃ | 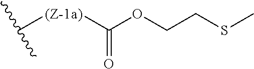 |
| 1.40 | F | F | (R)—CH₃ | 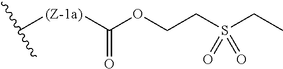 |
| 1.41 | F | F | (R)—CH₃ | 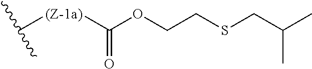 |
| 1.42 | F | F | (R)—CH₃ | 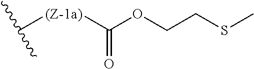 |
| 1.43 | F | CN | (R,S)—CF₃ | 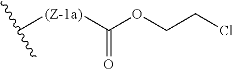 |
| 1.44 | F | CN | (R,S)—CF₃ | 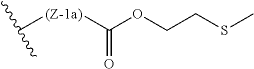 |
| 1.45 | Cl | CN | (R,S)—CH₃ | 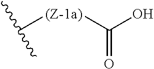 |
| 1.46 | F | F | (R,S)—CH₃ | 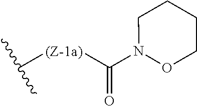 |
| 1.47 | F | F | (R,S)—CH₃ | 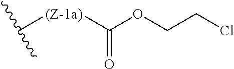 |
| 1.48 | F | F | (R,S)-vinyl | 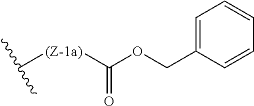 |
| 1.49 | F | F | (R,S)-vinyl | 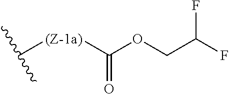 |
| 1.50 | F | F | (S)-vinyl | 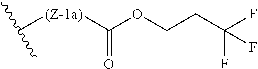 |
| 1.51 | F | H | (S)-vinyl | 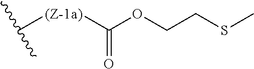 |
| 1.52 | F | F | (S)-vinyl | 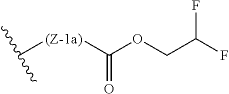 |
| 1.53 | F | Cl | (R,S)—CH₃ | 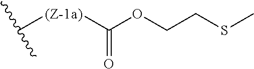 |
| 1.54 | F | F | (R)-cPr | 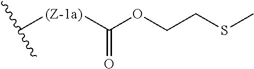 |
| 1.55 | F | F | (R)—CH₂Cl | 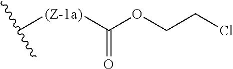 |
| 1.56 | F | F | (R)—CF₃ | 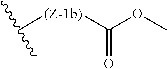 |
| 1.57 | F | F | (S)-vinyl | 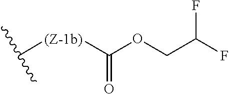 |
| 1.58 | F | F | (S)-vinyl | 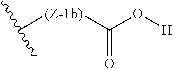 |
| 1.59 | F | H | (R,S)—CH₃ | 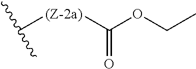 |
| 1.60 | F | F | (R,S)-vinyl | 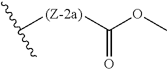 |
| 1.61 | F | F | (R,S)—CH₃ | 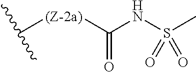 |
| 1.62 | F | F | (R,S)—CH₃ | 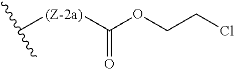 |
| 1.63 | F | F | (R)—CH₃ | 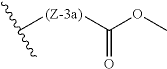 |
| 1.64 | F | F | (R)—CH₃ | 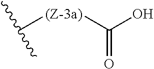 |
| 1.65 | F | CH₃ | (S)-vinyl | 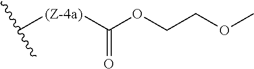 |
| 1.66 | F | F | (S)-vinyl | 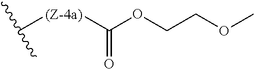 |
| 1.67 | F | H | (R,S)—CH₃ | 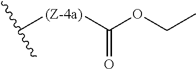 |
| 1.68 | F | F | (S)-vinyl | 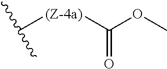 |
| 1.69 | F | F | (S)-vinyl | 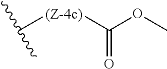 |
| 1.70 | F | F | (S)-vinyl | 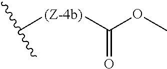 |
| 1.71 | F | CH₃ | (S)-vinyl | 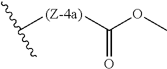 |
| 1.72 | F | F | (R,S)—OCH₃ | 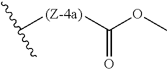 |
| 1.73 | F | F | (R)—CH₃ | 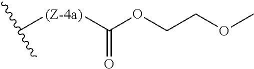 |
| 1.74 | F | F | (R)—CH₃ | 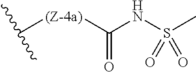 |
| 1.75 | F | F | (R)—CH₃ | 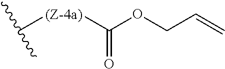 |
| 1.76 | F | F | (R)—CH₃ | 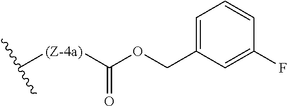 |
| 1.77 | F | H | (R,S)—CH₃ | 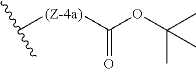 |
| 1.78 | F | H | (R,S)—CH₃ | 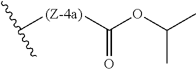 |
| 1.79 | F | H | (R,S)—CH₃ | 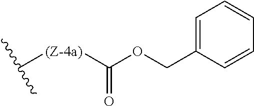 |
| 1.80 | F | F | (S)-vinyl | 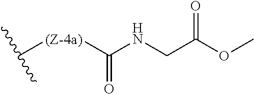 |
| 1.81 | F | F | (S)-vinyl | 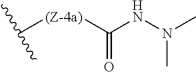 |
| 1.82 | F | F | (S)-vinyl | 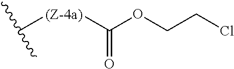 |
| 1.83 | F | F | (S)-vinyl | 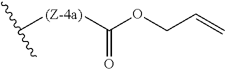 |
| 1.84 | F | F | (S)-vinyl | 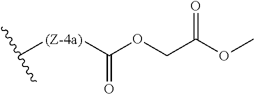 |
| 1.85 | F | CH₃ | (S)-vinyl | 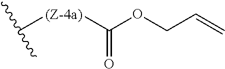 |
| 1.86 | F | CH₃ | (S)-vinyl | 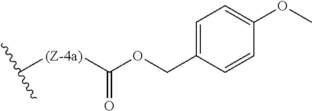 |
| 1.87 | F | F | (S)-vinyl | 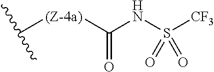 |
| 1.88 | F | F | (R)—CF₂CH₃ | 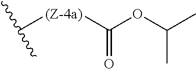 |
| 1.89 | F | CH₃ | (S)-vinyl | 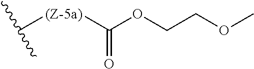 |
| 1.90 | CH₃ | CH₃ | (S)-vinyl | 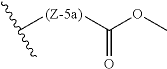 |
| 1.91 | F | F | (S)-vinyl | 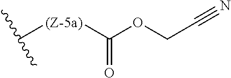 |
| 1.92 | F | F | (R)—CH₃ | 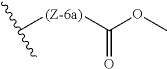 |
| 1.93 | F | F | (R,S)—CH₃ | 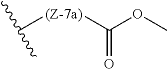 |
| 1.94 | F | F | (R)—CF₂CH₃ | 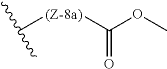 |

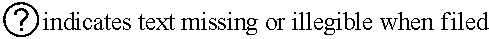

Surprisingly, it has now been found that the above-defined active compound combinations of substituted isoxazolincarboxamides of the general formula (I) and/or their salts and metcamifen, whilst being tolerated very well by crop plants, have particularly high herbicidal activity and can be used in various crops, in particular in cereal (especially wheat and barley) and maize, but also in soya beans, potatoes and rice, for the selective control of weeds (=undesirable plants).

Here, it has to be considered to be surprising that, from a large number of known safeners or antidotes which are capable of antagonizing the damaging effect of a herbicide on the crop plants, that it is metcamifen which neutralize the damaging effect of substituted isoxazolincarboxamides on the crop plants virtually completely without negatively affecting the herbicidal activity with respect to the weeds.

Emphasis is given here to the particularly advantageous effect of metcamifen, in particular in respect of sparing cereal plants, such as, for example, wheat, barley and rye, but also maize and rice, as crop plants.

The active compound combinations according to the invention can be used, for example, in connection with the following plants:

Dicotyledonous weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindemia, Lamium, Veronica, Abutilon, Emex, Datura, Viola, Galeopsis, Papaver, Centaurea, Trifolium, Ranunculus, Taraxacum.

Dicotyledonous crops of the genera: Gossypium, Glycine, Beta, Daucus, Phaseolus, Pisum, Solanum, Linum, Ipomoea, Vicia, Nicotiana, Lycopersicon, Arachis, Brassica, Lactuca, Cucumis, Cuburbita, Helianthus.

Monocotyledonous weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristylis, Sagittaria, Eleocharis, Scirpus, Paspalum, Ischaemum, Sphenoclea, Dactyloctenium, Agrostis, Alopecurus, Apera.

Monocotyledonous crops of the genera: Oryza, Zea, Triticum, Hordeum, Avena, Secale, Sorghum, Panicum, Saccharum, Ananas, Asparagus, Allium.

However, the use of the active compound combinations according to the invention is in no way restricted to these genera, but also extends in the same manner to other plants. According to the invention, crop plants are all plants and plant varieties including transgenic plants and plant varieties, where on transgenic plants and plant varieties it is also possible for synergistic effects to occur.

The invention furthermore relates to a method of reducing crop damage by treating the seed of the crop with the safener before sowing. This can be done in addition to the use of herbicide/safener combinations and compositions comprising thereof, which are highly suitable to protect crops from herbicide damage in pre- and post-emergence treatments.

It was an object of the present invention to provide a method for further reducing crop damage using known combinations of herbicides and safener and compositions comprising thereof. Surprisingly, this object is achieved by the following methods/schemes of treatment:

Method A

Step 1: Treatment of the seed with metcamifen

Step 2: Applying compound of formula (I) or compositions comprising thereof in a post-emergence treatment

Method B

Step 1: Treatment of the seed with metcamifen

Step 2: Applying compound of formula (I) or compositions comprising thereof in a pre-emergence treatment

Preferred are the following methods:

Method A-a

Step 1: Treatment of the seed with metcamifen

Step 2: Applying compound of formula (Ia) or compositions comprising thereof in a post-emergence treatment

Method B-a

Step 1: Treatment of the seed with metcamifen

Step 2: Applying compound of formula (Ia) or compositions comprising thereof in a pre-emergence treatment

Seed:

Seed of crop plants, such as, for example, various cereal species (such as wheat, triticale, barley, rye), corn, maize.

Composition

Compositions within the context of the present invention comprise in addition to the herbicide/safener combinations according to the invention one or more further component(s) which include, but are not limited to the following: formulation auxiliaries, additives customary in crop protection, further agrochemically active compounds (e.g. fungicides and insecticides).

Additives

Additives are for example, fertilizers and colorants.

Method 1 Day Before

The invention furthermore relates to a method of reducing crop damage by 24 hour pre incubation of the crop plant with the safener before applying the herbicide or herbicide/safener combination/composition.

In another embodiment the invention relates to selective herbicidal combinations comprising at least one further herbicide (c) wherein (c) is chosen from the list which includes but is not limited to the following:

Acetochlor, acifluorfen, acifluorfen-methyl, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, aminopyralid-dimethylammonium, aminopyralid-tripromine, amitrole, ammoniumsulfamate, anilofos, asulam, asulam-potassium, asulam sodium, atrazine, azafenidin, azimsulfuron, beflubutamid, (S)-(−)-beflubutamid, beflubutamid-M, benazolin, benazolin-ethyl, benazolin-dimethylammonium, benazolin-potassium, benfluralin, benfuresate, bensulfuron, bensulfuron-methyl, bensulide, bentazone, bentazone-sdium, benzobicyclon, benzofenap, bicyclopyrone, bifenox, bilanafos, bilanafos-sodium, bipyrazone, bispyribac, bispyribacsodium, bixlozone, bromacil, bromacil-lithium, bromacil-sodium, bromobutide, bromofenoxim, bromoxynil, bromoxynil-butyrate, -potassium, -heptanoate und -octanoate, busoxinone, butachlor, butafenacil, butamifos, butenachlor, butralin, butroxydim, butylate, cafenstrole, cambendichlor, carbetamide, carfentrazone, carfentrazone-ethyl, chloramben, chlorambenammonium, chloramben-diolamine, chlroamben-methyl, chloramben-methylammonium, chloramben-sodium, chlorbromuron, chlorfenac, chlorfenac-ammonium, chlorfenac-sodium, chlorfenprop, chlorfenprop-methyl, chlorflurenol, chlorflurenol-methyl, chloridazon, chlorimuron, chlorimuron-ethyl, chlorophthalim, chlorotoluron, chlorsulfuron, chlorthal, chlorthal-dimethyl, chlorthal-monomethyl, cinidon, cinidon-ethyl, cinmethylin, exo-(+)-cinmethylin, i.e. (1R,2S,4S)-4-isopropyl-1-methyl-2-[(2-methylbenzyl)oxy]-7-oxabicyclo[2.2.1]heptane, exo-(−)-cinmethylin, i.e. (1R,2S,4S)-4-isopropyl-1-methyl-2-[(2-methylbenzyl)oxy]-7-oxabicyclo[2.2.1]heptane, cinosulfuron, clacyfos, clethodim, clodinafop, clodinafop-ethyl, clodinafop-propargyl, clomazone, clomeprop, clopyralid, clopyralid-methyl, clopyralid-olamine, clopyralid-potassium, clopyralid-tripomine, cloransulam, cloransulammethyl, cumyluron, cyanamide, cyanazine, cycloate, cyclopyranil, cyclopyrimorate, cyclosulfamuron, cycloxydim, cyhalofop, cyhalofop-butyl, cyprazine, 2,4-D (including theammonium, butotyl, -butyl, choline, diethylammonium, -dimethylammonium, -diolamine, -doboxyl, -dodecylammonium, etexyl, ethyl, 2-ethylhexyl, heptylammonium, isobutyl, isooctyl, isopropyl, isopropylammonium, lithium, meptyl, methyl, potassium, tetradecylammonium, triethylammonium, triisopropanolammonium, tripromine and trolamine salt thereof), 2,4-DB, 2,4-DB-butyl, -dimethylammonium, isooctyl, -potassium und -sodium, daimuron (dymron), dalapon, dalapon-calcium, dalapon-magnesium, dalapon-sodium, dazomet, dazomet-sodium, ndecanol, 7-deoxy-D-sedoheptulose, desmedipham, detosyl-pyrazolate (DTP), dicamba and its salts, e. g. dicamba-biproamine, dicamba-N,N-Bis(3-aminopropyl)methylamine, dicambabutotyl, dicamba-choline, dicamba-diglycolamine, dicamba-dimethylammonium, dicambadiethanolaminemmonium, dicamba-diethylammonium, dicamba-isopropylammonium, dicamba-methyl, dicamba-monoethanolaminedicamba-olamine, dicamba-potassium, dicambasodium, dicamba-triethanolamine, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-butotyl, dichlroprop-dimethylammonium, dichhlorprop-etexyl, dichlorpropethylammonium, dichlorprop-isoctyl, dichlorprop-methyl, dichlorprop-postassium, dichlorpropsodium, dichlorprop-P, dichlorprop-P-dimethylammonium, dichlorprop-P-etexyl, dichlorpropP-potassium, dichlorprop-sodium, diclofop, diclofop-methyl, diclofop-P, diclofop-P-methyl, diclosulam, difenzoquat, difenzoquat-metilsulfate, diflufenican, diflufenzopyr, diflufenzopyrsodium, dimefuron, dimepiperate, dimesulfazet, dimethachlor, dimethametryn, dimethenamid, dimethenamid-P, dimetrasulfuron, dinitramine, dinoterb, dinoterb-acetate, diphenamid, diquat, diquat-dibromid, diquat-dichloride, dithiopyr, diuron, DNOC, DNOC-ammonium, DNOCpotassium, DNOC-sodium, endothal, endothal-diammonium, endothal-dipotassium, endothaldisodium, Epyrifenacil (S-3100), EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-5231, i.e. N-[2-Chlor-4-fluor-5-[4-(3-fluorpropyl)-4,5-dihydro-5-oxo-1H-tetrazol-1-yl]-phenyl]-ethansulfonamid, F-7967, i.e. 3-[7-Chlor-5-fluor-2-(trifluormethyl)-1H-benzimidazol-4-yl]-1-methyl-6-(trifluormethyl)pyrimidin-2,4(1H,3H)-dion, fenoxaprop, fenoxaprop-P, fenoxaprop-ethyl, fenoxaprop-P-ethyl, fenoxasulfone, fenpyrazone, fenquinotrione, fentrazamide, flamprop, flamprop-isoproyl, flamprop-methyl, flamprop-M-isopropyl, flamprop-M-methyl, flazasulfuron, florasulam, florpyrauxifen, florpyrauxifenbenzyl, fluazifop, fluazifop-butyl, fluazifop-methyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium und -methyl, fluoroglycofen, fluoroglycofenethyl, flupropanate, flupropanate-sdium, flupyrsulfuron, flupyrsulfuron-methyl, flupyrsulfuronmethyl-sodium, fluridone, flurochloridone, fluroxypyr, fluroxypyr-butometyl, fluroxypyrmeptyl, flurtamone, fluthiacet, fluthiacet-methyl, fomesafen, fomesafen-sodium, foramsulfuron, foramsulfuron sodium salt, fosamine, fosamine-ammonium, glufosinate, glufosinateammonium, glufosinate-sodium, L-glufosinate-ammonium, L-glufosiante-sodium, glufosinateP-sodium, glufosinate-P-ammonium, glyphosate, glyphosate-ammonium, -isopropylammonium, -diammonium, -dimethylammonium, -potassium, -sodium, sesquisodium and -trimesium, H-9201, i.e. O-(2,4-Dimethyl-6-nitrophenyl)-O-ethylisopropylphosphoramidothioat, halauxifen, halauxifen-methyl, halosafen, halosulfuron, halosulfuron-methyl, haloxyfop, haloxyfop-P, haloxyfop-ethoxyethyl, haloxyfop-Pethoxyethyl, haloxyfop-methyl, haloxyfop-P-methyl, haloxifop-sodium, hexazinone, HNPCA8169, i.e. prop-2-yn-1-yl (2S)-2-{3-[(5-tert-butylpyridin-2-yl)oxy]phenoxy}propanoate, HW02, i.e. 1-(Dimethoxyphosphoryl)-ethyl-(2,4-dichlorphenoxy)acetat, hydantocidin, imazamethabenz, imazamethabenz-methyl, imazamox, imazamox-ammonium, imazapic, imazapic-ammonium, imazapyr, imazapyr-isopropylammonium, imazaquin, imazaquinammonium, imazaquin.methyl, imazethapyr, imazethapyr-immonium, imazosulfuron, indanofan, indaziflam, iodosulfuron, iodosulfuron-methyl, iodosulfuron-methyl-sodium, ioxynil, ioxynil-lithium, -octanoate, -potassium und sodium, ipfencarbazone, isoproturon, isouron, isoxaben, isoxaflutole, karbutilate, KUH-043, i.e. 3-({[5-(Difluormethyl)-1-methyl-3-(trifluormethyl)-1H-pyrazol-4-yl]methyl}sulfonyl)-5,5-dimethyl-4,5-dihydro-1,2-oxazol, ketospiradox, ketospiradox-potassium, lactofen, lenacil, linuron, MCPA, MCPA-butotyl, -butyl, -dimethylammonium, -diolamine, -2-ethylhexyl, -ethyl, -isobutyl, isoctyl, -isopropyl, -isopropylammonium, -methyl, olamine, -potassium, -sodium and -trolamine, MCPB, MCPBmethyl, -ethyl und -sodium, mecoprop, mecoprop-butotyl, mecoprop-demethylammonium, mecoprop-diolamine, mecoprop-etexyl, mecoprop-ethadyl, mecoprop-isoctyl, mecopropmethyl, mecoprop-potassium, mecoprop-sodium, and mecoprop-trolamine, mecoprop-P, mecoprop-P-butotyl, -dimethylammonium, -2-ethylhexyl and -potassium, mefenacet, mefluidide, mefluidide-diolamine, mefluidide-potassium, mesosulfuron, mesosulfuron-methyl, mesosulfuron sodium salt, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuronmethyl, MT-5950, i.e. N-[3-chlor-4-(1-methylethyl)-phenyl]-2-methylpentanamid, NGGC-011, napropamide, NC-310, i.e. 4-(2,4-Dichlorbenzoyl)-1-methyl-5-benzyloxypyrazol, NC-656, i.e. 3-[(isopropylsulfonyl)methyl]-N-(5-methyl-1,3,4-oxadiazol-2-yl)-5-(trifluoromethyl)[1,2,4]triazolo[4,3-a]pyridine-8-carboxamide, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefone, oxyfluorfen, paraquat, paraquatdichloride, paraquat-dimethylsulfate, pebulate, pendimethalin, penoxsulam, pentachlorphenol, pentoxazone, pethoxamid, petroleum oils, phenmedipham, phenmedipham-ethyl, picloram, picloram-dimethylammonium, picloram-etexyl, picloram-isoctyl, picloram-methyl, picloramolamine, picloram-potassium, picloram-triethylammonium, picloram-tripromine, picloramtrolamine, picolinafen, pinoxaden, piperophos, pretilachlor, primisulfuron, primisulfuronmethyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufenethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quincloracdimethylammonium, quinclorac-methyl, quinmerac, quinoclamine, quizalofop, quizalofopethyl, quizalofop-P, quizalofop-P-ethyl, quizalofop-P-tefuryl, QYM201, i.e. 1-{2-chloro-3-[(3-cyclopropyl-5-hydroxy-1-methyl-1H-pyrazol-4-yl)carbonyl]-6-(trifluoromethyl)phenyl}piperidin-2-one, rimsulfuron, saflufenacil, sethoxydim, siduron, simazine, simetryn, SL-261, sulcotrione, sulfentrazone, sulfometuron, sulfometuron-methyl, sulfosulfuron, SYP-249, i.e. 1-Ethoxy-3-methyl-1-oxobut-3-en-2-yl-5-[2-chlor-4-(trifluormethyl)phenoxy]-2-nitrobenzoat, SYP-300, i.e. 1-[7-Fluor-3-oxo-4-(prop-2-in-1-yl)-3,4-dihydro-2H-1,4-benzoxazin-6-yl]-3-propyl-2-thioxoimidazolidin-4,5-dion, 2,3,6-TBA, TCA (trichloro acetic acid) and its salts, e.g. TCA-ammonium, TCA-calcium, TCA-ethyl, TCA-magnesium, TCA-sodium, tebuthiuron, tefuryltrione, tembotrione, tepraloxydim, terbacil, terbucarb, terbumeton, terbuthylazine, terbutryn, tetflupyrolimet, thaxtomin, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, triclopyr-butotyl, triclopyrcholine, triclopyr-ethyl, triclopyr-triethylammonium, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-Dichlor-N-{2-[(4,6-dimethoxypyrimidin-2-yl)oxy]benzyl}anilin, 3-(2-chloro-4-fluoro-5-(3-methyl-2,6-dioxo-4-trifluoromethyl-3,6-dihydropyrimidin-1 (2H)-yl)phenyl)-5-methyl-4,5-dihydroisoxazole-5-carboxylic acid ethyl ester, 3-chloro-2-[3-(difluoromethyl)isoxazolyl-5-yl]phenyl-5-chloropyrimidin-2-yl ether, 2-(3,4-dimethoxyphenyl)-4-[(2-hydroxy-6-oxocyclohex-1-en-1-yl)carbonyl]-6-methylpyridazine-3(2H)-one, 2-({2-[(2-methoxyethoxy)methyl]-6-methylpyridin-3-yl}carbonyl)cyclohexane-1,3-dione, (5-hydroxy-1-methyl-1H-pyrazol-4-yl)(3,3,4-trimethyl-1,1-dioxido-2,3-dihydro-1-benzothiophen-5-yl)methanone, 1-methyl-4-[(3,3,4-trimethyl-1,1-dioxido-2,3-dihydro-1-benzothiophen-5-yl)carbonyl]-1H-pyrazol-5-yl propane-1-sulfonate, 4-{2-chloro-3-[(3,5-dimethyl-1H-pyrazol-1-yl)methyl]-4-(methylsulfonyl)benzoyl}-1-methyl-1H-pyrazol-5-yl 1,3-dimethyl-1H-pyrazole-4-carboxylate; cyanomethyl 4-amino-3-chloro-5-fluoro-6-(7-fluoro-1H-indol-6-yl)pyridine-2-carboxylate, prop-2-yn-1-yl 4-amino-3-chloro-5-fluoro-6-(7-fluoro-1H-indol-6-yl)pyridine-2-carboxylate, methyl 4-amino-3-chloro-5-fluoro-6-(7-fluoro-1H-indol-6-yl)pyridine-2-carboxylate, 4-amino-3-chloro-5-fluoro-6-(7-fluoro-1H-indol-6-yl)pyridine-2-carboxylic acid, benzyl 4-amino-3-chloro-5-fluoro-6-(7-fluoro-1H-indol-6-yl)pyridine-2-carboxylate, ethyl 4-amino-3-chloro-5-fluoro-6-(7-fluoro-1H-indol-6-yl)pyridine-2-carboxylate, methyl 4-amino-3-chloro-5-fluoro-6-(7-fluoro-1-isobutyryl-1H-indol-6-yl)pyridine-2-carboxylate, methyl 6-(1-acetyl-7-fluoro-1H-indol-6-yl)-4-amino-3-chloro-5-fluoropyridine-2-carboxylate, methyl 4-amino-3-chloro-6-[1-(2,2-dimethylpropanoyl)-7-fluoro-1H-indol-6-yl]-5-fluoropyridine-2-carboxylate, methyl 4-amino-3-chloro-5-fluoro-6-[7-fluoro-1-(methoxyacetyl)-1H-indol-6-yl]pyridine-2-carboxylate, potassium 4-amino-3-chloro-5-fluoro-6-(7-fluoro-1H-indol-6-yl)pyridine-2-carboxylate, sodium 4-amino-3-chloro-5-fluoro-6-(7-fluoro-1H-indol-6-yl)pyridine-2-carboxylate, butyl 4-amino-3-chloro-5-fluoro-6-(7-fluoro-1H-indol-6-yl)pyridine-2-carboxylate, 4-hydroxy-1-methyl-3-[4-(trifluoromethyl)pyridin-2-yl]imidazolidin-2-one, 3-(5-tert-butyl-1,2-oxazol-3-yl)-4-hydroxy-1-methylimidazolidin-2-one,

abscisic acid, acibenzolar, acibenzolar-S-methyl, 1-aminocyclopro-1-yl carboxylic acid and derivatives thereof, 5-Aminolivulinsiure, ancymidol, 6-benzylaminopurine, brassinolide, brassinolide-ethyl, catechin, chitooligosaccharides (CO; COs differ from LCOs in that they lack the pendant fatty acid chain that is characteristic of LCOs. COs, sometimes referred to as N-acetylchitooligosaccharides, are also composed of GlcNAc residues but have side chain decorations that make them different from chitin molecules [(C₈H₁₃NO₅)ₙ, CAS No. 1398-61-4] and chitosan molecules [(C₅H₁₁NO₄)ₙ, CAS No. 9012-76-4]), chitinous compounds, chlormequat chloride, cloprop, cyclanilide, 3-(Cycloprop-1-enyl)propionic acid, daminozide, dazomet, dazomet-sodium, n-decanol, dikegulac, dikegulac-sodium, endothal, endothaldipotassium, -disodium, and mono(N,N-dimethylalkylammonium), ethephon, flumetralin, flurenol, flurenol-butyl, flurenol-methyl, flurprimidol, forchlorfenuron, gibberellic acid, inabenfide, indol-3-acetic acid (IAA), 4-indol-3-ylbutyric acid, isoprothiolane, probenazole, jasmonic acid, Jasmonic acid or derivatives thereof (e.g. Jasmonic acid methyl ester), lipochitooligosaccharides (LCO, sometimes referred to as symbiotic nodulation (Nod) signals (or Nod factors) or as Myc factors, consist of an oligosaccharide backbone of β-1,4-linked N-acetyl-D-glucosamine (“GlcNAc”) residues with an N-linked fatty acyl chain condensed at the non-reducing end. As understood in the art, LCOs differ in the number of GlcNAc residues in the backbone, in the length and degree of saturation of the fatty acyl chain and in the substitutions of reducing and non-reducing sugar residues), linoleic acid or derivatives thereof, linolenic acid or derivatives thereof, maleic hydrazide, mepiquat chloride, mepiquat pentaborate, 1-methylcyclopropene, 3′-methyl abscisic acid, 2-(1-naphthyl)acetamide, 1-naphthylacetic acid, 2-naphthyloxyacetic acid, nitrophenolate-mixture, 4-Oxo-4[(2-phenylethyl)amino]butyric acid, paclobutrazol, 4-phenylbutyric acid, N-phenylphthalamic acid, prohexadione, prohexadione-calcium, prohydrojasmon, salicylic acid, salicylic acid methyl ester, strigolacton, tecnazene, thidiazuron, triacontanol, trinexapac, trinexapac-ethyl, tsitodef, uniconazole, uniconazole-P, 2-fluoro-N-(3-methoxyphenyl)-9H-purin-6-amine.

The active compounds or active compound combinations/compositions can be converted into the customary formulations, such as solutions, emulsions, wettable powders, suspensions, powders, dusting agents, pastes, soluble powders, granules, suspoemulsion concentrates, natural and synthetic materials impregnated with active compound, and very fine capsules in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers.

If the extender used is water, it is also possible to use, for example, organic solvents as auxiliary solvents. Suitable liquid solvents are essentially: aromatics, such as xylene, toluene or alkylnaphthalenes, chlorinated aromatics and chlorinated aliphatic hydrocarbons, such as chlorobenzenes, chloroethylenes or methylene chloride, aliphatic hydrocarbons, such as cyclohexane or paraffins, for example petroleum fractions, mineral and vegetable oils, alcohols, such as butanol or glycol, and also their ethers and esters, ketones, such as acetone, methyl ethyl ketone, methyl isobutyl ketone or cyclohexanone, strongly polar solvents, such as dimethylformamide and dimethyl sulphoxide, and also water.

Suitable solid carriers are:

for example ammonium salts and ground natural minerals, such as kaolins, clays, talc, chalk, quartz, attapulgite, montmorillonite or diatomaceous earth, ground synthetic minerals, such as finely divided silica, alumina and silicates, suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise from 0.1 to 95 percent by weight of active compounds including the safeners, preferably between 0.5 and 90%.

The active compound combinations according to the invention are generally used in the form of finished formulations. However, the active compounds contained in the active compound combinations can also be mixed in individual formulations when used, i.e. in the form of tank mixes.

The novel active compound combinations, as such or in their formulations, can furthermore be used as a mixture with other known herbicides, finished formulations or tank mixes again being possible. A mixture with other known active compounds, such as fungicides, insecticides, acaricides, nematicides, bird repellents, growth factors, plant nutrients and agents which improve soil structure, is also possible. For certain intended uses, in particular in the post-emergence method, it may furthermore be advantageous to include, as further additives in the formulations, mineral or vegetable oils which are tolerated by plants (for example the commercial preparation “Rako Binol”), or ammonium salts such as, for example, ammonium sulphate or ammonium thiocyanate.

The novel active compound combinations can be used as such, in the form of their formulations or the use forms prepared therefrom by further dilution, such as ready-to-use solutions, suspensions, emulsions, powders, pastes and granules. They are used in the customary manner, for example by washing, spraying, atomizing, dusting or scattering.

The advantageous effect of the crop plant compatibility of the active compound combinations/compositions according to the invention is particularly highly pronounced at certain amounts of herbicide and safener.

The amounts of the active compound combinations according to the invention applied can be varied within a certain range; they depend, inter alia, on the weather and on soil factors.

In general, the application rates of the herbicide are between 0.1 and 1000 g per ha, preferably between 0.1 and 10 g per ha.

In general, the application rates of the safener are between 1 and 1000 g per ha, preferably between 10 and 200 g per ha.

In seed treatment applications the application rates of the safener are between 0.01 and 2 g per kg seed, preferably between 0.1 and 1 g per kg seed.

The active compound combinations/compositions according to the invention can be applied before and after emergence of the plants, that is to say by the pre-emergence and post-emergence method.

### USE EXAMPLES

Tank-Mix; Post-Emergence

A) Method Description

Seeds of crops (spring wheat, TRZAS; spring barley, HORVS; maize, ZEAMA) were placed in sandy loam soil in peat pots, covered with soil and cultivated in a greenhouse under good growth conditions. Two to three weeks after sowing, the test plants were treated at the 1 to 3-leaf stage. The herbicide/safener active compound combinations according to the invention, formulated as wettable powders or emulsion concentrates, and, in parallel tests, the correspondingly formulated individual active compounds were sprayed onto the green parts of the plants in various dosages using an amount of water of 300 l/ha (converted).

The pots were returned under good growing conditions in a glasshouse and a visual assessment of herbicidal effects was made at intervals from 1 to 3 weeks after herbicide application (DAT=days after treatment). Assessment was on a percentage basis in comparison with untreated control plants (0%=no injury, 100%=complete kill).

The effectiveness of the safener treatment is displayed as: Reduction [Difference]=herbicide damage without safener−herbicide damage with safener Reduction [%]=(Reduction [Difference]*100)/herbicide damage without safener

B) Tables with Data

a) Plant Species, Variety: ZEAMA Sileno, Safener (SAF): Metcamifen

TABLE 2

| Herbicide | | HER | | | HER | | |
|---|---|---|---|---|---|---|---|
| (HER) | | dose | | SAF | effect | | |
| [Exam- | | [g | HER | dose | + | | Reduc- |
| ple | Assess- | a.i./ | effect | [g | SAF | Reduction | tion |
| No.] | ment | ha] | [%] | a.i./ha] | [%] | [Difference] | [%] |
| 1.26 | Final | 32 | 94 | 100 | 35 | 59 | 63 |
| 1.26 | Final | 8 | 57 | 50 | 22 | 35 | 61 |
| 1.68 | Final | 32 | 98 | 50 | 67 | 31 | 32 |
| 1.68 | Final | 8 | 96 | 100 | 48 | 48 | 50 |

b) Plant Species, Variety: HORVS Montoya, Safener (SAF): Metcamifen

TABLE 3

| Herbicide | | HER | | | HER | | |
|---|---|---|---|---|---|---|---|
| (HER) | | dose | | SAF | effect | | |
| [Exam- | Assess- | [g | HER | dose | + | | Reduc- |
| ple | ment | a.i./ | effect | [g | SAF | Reduction | tion |
| No.] | [DAT] | ha] | [%] | a.i./ha] | [%] | [Difference] | [%] |
| 1.26 | 10 DAT | 2 | 70 | 50 | 50 | 20 | 29 |
| 1.26 | 10 DAT | 0.5 | 40 | 50 | 10 | 30 | 75 |

c) Plant Species, Variety: TRZAS Triso, Safener (SAF): Metcamifen

TABLE 4

| Herbicide | | HER | | | HER | | |
|---|---|---|---|---|---|---|---|
| (HER) | | dose | | SAF | effect | | |
| [Exam- | Assess- | [g | HER | dose | + | | Reduc- |
| ple | ment | a.i./ | effect | [g | SAF | Reduction | tion |
| No.] | [DAT] | ha] | [%] | a.i./ha] | [%] | [Difference] | [%] |
| 1.26 | 21 DAT | 2 | 90 | 50 | 80 | 10 | 11 |
| 1.26 | 21 DAT | 0.5 | 40 | 50 | 20 | 20 | 50 |

Seed Treatment; Pre-Emergence

A) Method Description

For the safener seed treatment, sufficient seeds of the respective crops (spring wheat, TRZAS; spring barley, HORVS; maize, ZEAMA) were weighed into screw top glass bottles approximately twice the volume of the seeds.

The specified safeners, formulated as wettable powder (WP) were weighed out so that the specified rates (g a.i./kg seed) would be obtained, dissolved in water (1 ml water per 10 g of seeds), and added to the seeds to produce a slurry.

The bottles were capped and then placed in an overhead shaker (set at medium speed for ca. 60 minutes) so that the seeds were evenly coated with the slurry. The bottles were uncapped and the seeds were either placed on paper and dried for an interval of 3-4 hours prior to sowing, or directly sown. Seeds were placed in sandy loam soil in pots of a diameter of 7-8 cm, and covered with soil.

Pre-emergence application of the specified herbicides was done subsequently, on two sets of plants:

a) seed treatment with safener, as described above

b) no safener treatment

The herbicides were formulated as WP and was sprayed onto the soil surface as an aqueous suspension at an equivalent water application rate of 300 l/ha.

After application, the test plants were kept in the greenhouse under good growth conditions. At intervals up to 4 weeks after application (=28 days after treatment; DAT), % crop damage observed on the treated plants was scored visually in comparison to control plants that had not received any safener or herbicide treatment.

Values in the table below are mean values of at least 2 replicates. The effectiveness of the safener treatment is displayed as: Reduction [Difference]=herbicide damage without safener−herbicide damage with safener Reduction [%]=(Reduction [Difference]*100)/herbicide damage without safener

B) Tables with Data

a) Plant Species, Variety: TRZAS Triso, Safener (SAF): Metcamifen

TABLE 5

| Herbicide | | HER | | SAF | HER | | |
|---|---|---|---|---|---|---|---|
| (HER) | | dose | | dose | effect | | |
| [Exam- | Assess- | [g | HER | [g | + | | Reduc- |
| ple | ment | a.i./ | effect | a.i./kg | SAF | Reduction | tion |
| No.] | [DAT] | ha] | [%] | seed] | [%] | [Difference] | [%] |
| 1.68 | 21 (Final) | 5 | 45 | 0.25 | 10 | 35 | 78 |
| 1.93 | 21 (Final) | 15 | 65 | 0.25 | 10 | 55 | 85 |
| 1.93 | 21 (Final) | 5 | 35 | 0.25 | 5 | 30 | 86 |

b) Plant Species, Variety: HORVS Montoya, Safener (SAF): Metcamifen

TABLE 6

| Herbicide | | HER | | SAF | HER | | |
|---|---|---|---|---|---|---|---|
| (HER) | | dose | | dose | effect | | |
| [Exam- | Assess- | [g | HER | [g | + | | Reduc- |
| ple | ment | a.i./ | effect | a.i./kg | SAF | Reduction | tion |
| No.] | [DAT] | ha] | [%] | seed] | [%] | [Difference] | [%] |
| 1.18 | 21 (Final) | 15 | 40 | 0.25 | 20 | 20 | 50 |

c) Plant Species, Variety: ZEAMA Sileno, Safener (SAF): Metcamifen

TABLE 7

| Herbicide | | HER | | SAF | HER | | |
|---|---|---|---|---|---|---|---|
| (HER) | | dose | | dose | effect | | |
| [Exam- | Assess- | [g | HER | [g | + | | Reduc- |
| ple | ment | a.i./ | effect | a.i./kg | SAF | Reduction | tion |
| No.] | [DAT] | ha] | [%] | seed] | [%] | [Difference] | [%] |
| 1.68 | 14 | 80 | 97 | 0.25 | 75 | 22 | 23 |
| 1.68 | 14 | 40 | 93 | 0.25 | 30 | 63 | 68 |
| 1.68 | 26 | 40 | 95 | 0.25 | 25 | 70 | 74 |
| 1.70 | 14 | 80 | 95 | 0.25 | 35 | 60 | 63 |
| 1.70 | 26 | 80 | 95 | 0.25 | 25 | 70 | 74 |
| 1.70 | 14 | 40 | 80 | 0.25 | 20 | 60 | 75 |
| 1.70 | 26 | 40 | 85 | 0.25 | 5 | 80 | 94 |
| 1.69 | 14 | 40 | 95 | 0.25 | 20 | 75 | 79 |
| 1.69 | 26 | 40 | 90 | 0.25 | 30 | 60 | 67 |
| 1.92 | 14 | 40 | 75 | 0.25 | 10 | 65 | 87 |
| 1.92 | 26 | 40 | 70 | 0.25 | 0 | 70 | 100 |
| 1.90 | 14 | 80 | 25 | 0.25 | 5 | 20 | 80 |
| 1.90 | 26 | 80 | 30 | 0.25 | 5 | 25 | 83 |
| 1.26 | 14 | 80 | 70 | 0.25 | 10 | 60 | 86 |
| 1.26 | 26 | 80 | 70 | 0.25 | 10 | 60 | 86 |
| 1.26 | 14 | 40 | 25 | 0.25 | 5 | 20 | 80 |
| 1.26 | 26 | 40 | 50 | 0.25 | 5 | 45 | 90 |
| 1.2 | 14 | 80 | 45 | 0.25 | 10 | 35 | 78 |
| 1.2 | 14 | 40 | 40 | 0.25 | 20 | 20 | 50 |
| 1.18 | 14 | 40 | 40 | 0.25 | 5 | 35 | 88 |
| 1.63 | 14 | 40 | 60 | 0.25 | 30 | 30 | 50 |
| 1.63 | 26 | 40 | 25 | 0.25 | 0 | 25 | 100 |
| 1.5 | 14 | 80 | 60 | 0.25 | 35 | 25 | 42 |
| 1.5 | 26 | 80 | 45 | 0.25 | 25 | 20 | 44 |
| 1.7 | 14 | 40 | 83 | 0.25 | 55 | 28 | 34 |
| 1.7 | 26 | 40 | 45 | 0.25 | 25 | 20 | 44 |

1. A combination comprising
(a) substituted isoxazolincarboxamide of formula (I) or agrochemical acceptable salt thereof 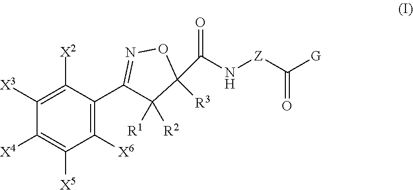
in which
G represents OR⁴ or NR⁷R⁸
R¹ and R² each represent hydrogen;
R³ represents (C₁-C₅)-alkyl, (C₃-C₆)-cycloalkyl, (C₂-C₅)-alkenyl, (C₂-C₅)-alkinyl or (C₁-C₅)-alkoxy each optionally substituted “m” times by substituents from the group consisting of halogen, cyano, (C₁-C₅)-alkoxy and hydroxy;
R⁴ represents hydrogen,
or
represents (C₁-C₁₂)-alkyl, (C₃-C₇)-cycloalkyl, (C₃-C₇)-cycloalkyl-(C₁-C₈)-alkyl, (C₂-C₈)-alkenyl, (C₅-C₅)-cycloalkenyl, (C₁-C₄)-alkylphenyl or (C₂-C₈)-alkinyl each optionally substituted “m” times by substituents from the group consisting of halogen, cyano, (C₁-C₆)-alkoxy, (C₁-C₆)-alkoxycarbonyl, hydroxy, S(O)ₙR⁵;
R⁵ represents (C₁-C₈)-alkyl, (C₂-C₈)-alkenyl, (C₃-C₆)-cycloalkyl, benzyl, CON((C₁-C₃)alkyl)₂ or (C₁-C₈)-alkyl-C(O)—(C₁-C₈)-alkyl each optionally substituted “m” times by substituents from the group consisting of halogen and cyano;
R⁶ represents hydrogen,
or
represents (C₁-C₈)-alkyl, (C₃-C₆)-cycloalkyl, (C₃-C₈)-alkenyl or (C₃-C₈)-alkinyl each optionally substituted “m” times by substituents from the group consisting of halogen, cyano und (C₁-C₂)-alkoxy;
R⁷, R⁸ independently of each other represent hydrogen, (C₁-C₆)-alkoxycarbonyl(C₁-C₆)-alkyl, N((C₁-C₃)-alkyl)₂, S(O)ₙR⁵,
or
R⁷ and R⁸ together with the nitrogen atom to which they are attached form a saturated or partially or fully unsaturated five-, six-, or seven-membered ring which may contain apart from the nitrogen atom “r” carbon atoms, “o” oxygen atoms and is optionally substituted “m” times by substituents from the group consisting of halogen, (C₁-C₆)-alkyl, halogen-(C₁-C₆)-alkyl, oxo, CO₂R⁶;
Z represents Z-1 to Z-8: 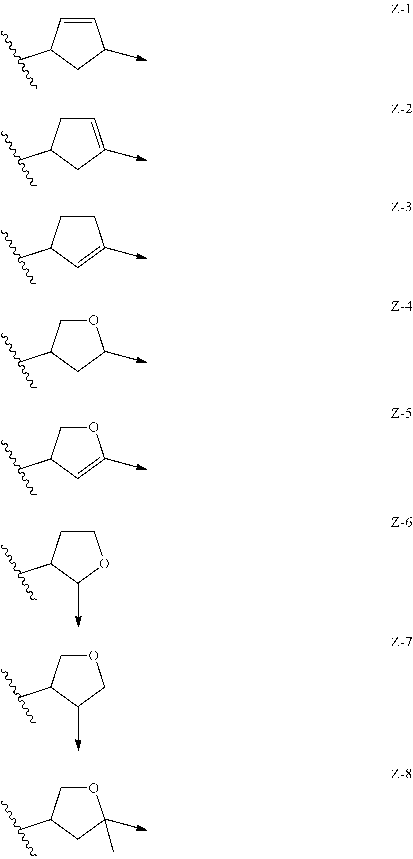
whereas the arrow represents the bonding to the group CO-G of formula (I);
X², X⁴ and X⁶ independently of one another represent hydrogen or fluorine;
X³ and X⁵ independently of one another represent hydrogen, chlorine, cyano or fluorine;
or
represents (C₁-C₃)-Alkyl, (C₁-C₃)-Alkoxy each optionally substituted “m” times by substituents from the group consisting of fluorine or chlorine;
m represents 0, 1, 2, 3, 4 or 5;
n represents 0, 1 or 2;
o represents 0, 1 or 2;
r represents 3, 4, 5 or 6;
and
(b) metcamifen.

2. The combination according to claim 1, wherein compound of formula (I) is (Ia) or an agrochemical acceptable salt thereof 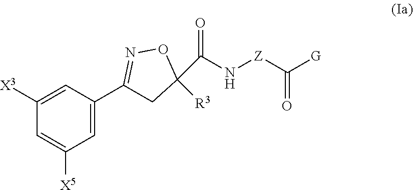
in which
X³, X⁵, R³ and G are as described above;
Z means Z-1a, Z-1 b, Z-2a, Z-3a, Z-4a, Z-5a, Z-6a, Z-7a, Z-8a, 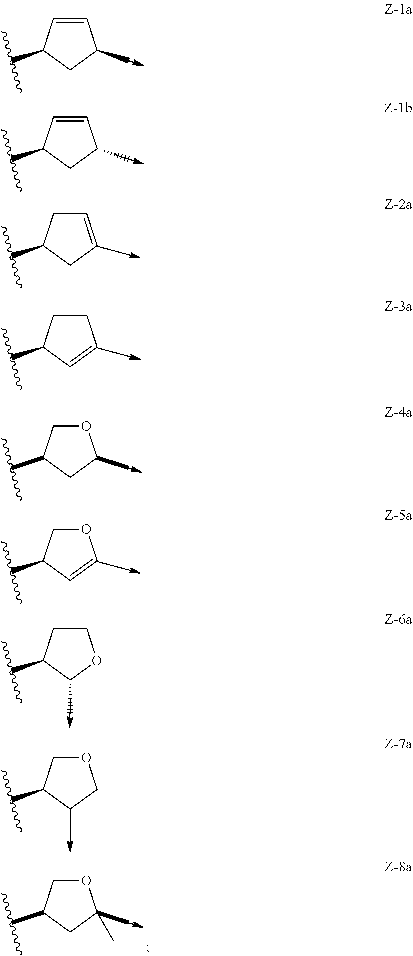
wherein Z-4a means the mixture of both structures Z-4b and Z-4c; 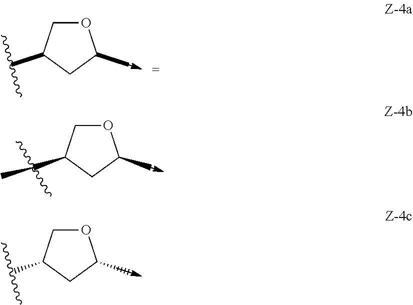
and wherein Z-8a means the mixture of both structures Z-8b and Z-8c 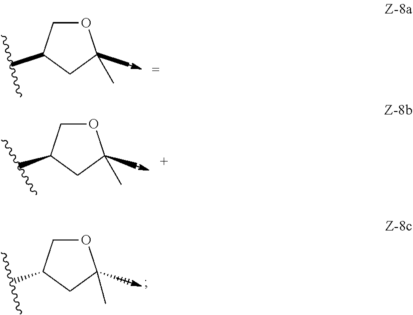
and wherein the arrow means a bond to the group CO-G in formula (Ia).

3. The combination according to claim 1 wherein the application rate of the compound of formula (I) or salt is adapted to be between 0.1 and 1000 g per ha, optionally between 0.1 and 10 g per ha and wherein the application rate of the metcamifen is adapted to be between 1 and 1000 g per ha, optionally between 10 and 200 g per ha.

4. A product comprising a combination according to claim 1 for controlling one or more undesirable plants.

5. A method for controlling one or more plants, wherein a combination according to claim 1 is allowed to act on one or more undesirable plants and/or a habitat thereof.

6. A composition comprising in addition to a combination according to claim 1, one or more surfactants and/or extenders.

7. A process for preparing a herbicidal composition according to claim 6, whereas a combination according to claim 1 is mixed with one or more surfactants and/or extenders.

8. A method of reducing crop damage comprising treating seed of a crop with metcamifen before sowing and applying a compound of formula (I) 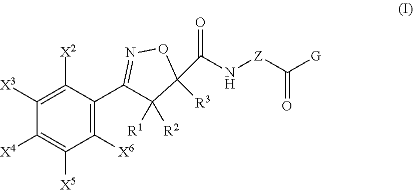
in which
G represents OR⁴ or NR⁷R⁸
R¹ and R² each represent hydrogen;
R³ represents (C₁-C₅)-alkyl, (C₃-C₆)-cycloalkyl, (C₂-C₅)-alkenyl, (C₂-C₅)-alkinyl or (C₁-C₅)-alkoxy each optionally substituted “m” times by substituents from the group consisting of halogen, cyano, (C₁-C₅)-alkoxy and hydroxy;
R⁴ represents hydrogen,
or
represents (C₁-C₁₂)-alkyl, (C₃-C₇)-cycloalkyl, (C₃-C₇)-cycloalkyl-(C₁-C₈)-alkyl, (C₂-C₈)-alkenyl, (C₅-C₆)-cycloalkenyl, (C₁-C₄)-alkylphenyl or (C₂-C₈)-alkinyl each optionally substituted “m” times by substituents from the group consisting of halogen, cyano, (C₁-C₆)-alkoxy, (C₁-C₆)-alkoxycarbonyl, hydroxy, S(O)ₙR⁵;
R⁵ represents (C₁-C₈)-alkyl, (C₂-C₈)-alkenyl, (C₃-C₆)-cycloalkyl, benzyl, CON((C₁-C₃)alkyl)₂ or (C₁-C₈)-alkyl-C(O)—(C₁-C₈)-alkyl each optionally substituted “m” times by substituents from the group consisting of halogen and cyano;
R⁶ represents hydrogen,
or
represents (C₁-C₈)-alkyl, (C₃-C₆)-cycloalkyl, (C₃-C₈)-alkenyl or (C₃-C₈)-alkinyl each optionally substituted “m” times by substituents from the group consisting of halogen, cyano und (C₁-C₂)-alkoxy;
R⁷, R⁸ independently of each other represent hydrogen, (C₁-C₆)-alkoxycarbonyl(C₁-C₆)-alkyl, N((C₁-C₃)-alkyl)₂, S(O)ₙR⁵,
or
R⁷ and R⁸ together with the nitrogen atom to which they are attached form a saturated or partially or fully unsaturated five-, six-, or seven-membered ring which may contain apart from the nitrogen atom “r” carbon atoms, “o” oxygen atoms and is optionally substituted “m” times by substituents from the group consisting of halogen, (C₁-C₆)-alkyl, halogen-(C₁-C₆)-alkyl, oxo, CO₂R⁶;
Z represents Z-1 to Z-8: 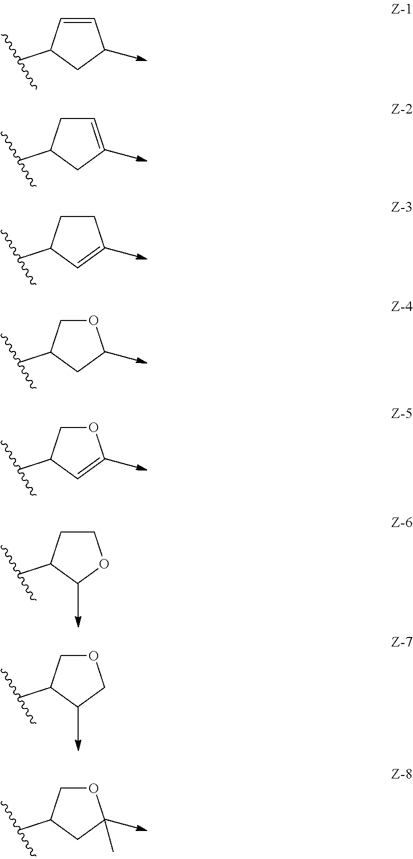
whereas the arrow represents the bonding to the group CO-G of formula (I);
X², X⁴ and X⁶ independently of one another represent hydrogen or fluorine;
X³ and X⁵ independently of one another represent hydrogen, chlorine, cyano or fluorine;
or
represents (C₁-C₃)-Alkyl, (C₁-C₃)-Alkoxy each optionally substituted “m” times by substituents from the group consisting of fluorine or chlorine;
m represents 0, 1, 2, 3, 4 or 5;
n represents 0, 1 or 2;
o represents 0, 1 or 2;
r represents 3, 4, 5 or 6;
or a salt or a combination/composition thereof in a post-emergence treatment.

9. A method of reducing crop damage comprising treating seed of a crop with metcamifen before sowing and applying a compound of formula (I) 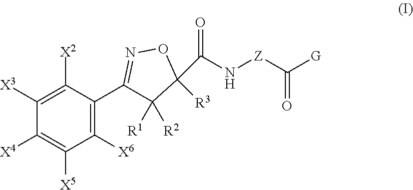
in which
G represents OR⁴ or NR⁷R⁸
R¹ and R² each represent hydrogen;
R³ represents (C₁-C₅)-alkyl, (C₃-C₆)-cycloalkyl, (C₂-C₅)-alkenyl, (C₂-C₅)-alkinyl or (C₁-C₅)-alkoxy each optionally substituted “m” times by substituents from the group consisting of halogen, cyano, (C₁-C₅)-alkoxy and hydroxy;
R⁴ represents hydrogen,
or
represents (C₁-C₁₂)-alkyl, (C₃-C₇)-cycloalkyl, (C₃-C₇)-cycloalkyl-(C₁-C₈)-alkyl, (C₂-C₈)-alkenyl, (C₅-C₆)-cycloalkenyl, (C₁-C₄)-alkylphenyl or (C₂-C₈)-alkinyl each optionally substituted “m” times by substituents from the group consisting of halogen, cyano, (C₁-C₆)-alkoxy, (C₁-C₆)-alkoxycarbonyl, hydroxy, S(O)ₙR⁵;
R⁵ represents (C₁-C₈)-alkyl, (C₂-C₈)-alkenyl, (C₃-C₆)-cycloalkyl, benzyl, CON((C₁-C₃)alkyl)₂ or (C₁-C₈)-alkyl-C(O)—(C₁-C₈)-alkyl each optionally substituted “m” times by substituents from the group consisting of halogen and cyano;
R⁶ represents hydrogen,
or
represents (C₁-C₈)-alkyl, (C₃-C₆)-cycloalkyl, (C₃-C₈)-alkenyl or (C₃-C₈)-alkinyl each optionally substituted “m” times by substituents from the group consisting of halogen, cyano und (C₁-C₂)-alkoxy;
R⁷, R⁸ independently of each other represent hydrogen, (C₁-C₆)-alkoxycarbonyl(C₁-C₆)-alkyl, N((C₁-C₃)-alkyl)₂, S(O)ₙR⁵,
or
R⁷ and R⁸ together with the nitrogen atom to which they are attached form a saturated or partially or fully unsaturated five-, six-, or seven-membered ring which may contain apart from the nitrogen atom “r” carbon atoms, “o” oxygen atoms and is optionally substituted “m” times by substituents from the group consisting of halogen, (C₁-C₆)-alkyl, halogen-(C₁-C₆)-alkyl, oxo, CO₂R⁶;
Z represents Z-1 to Z-8: 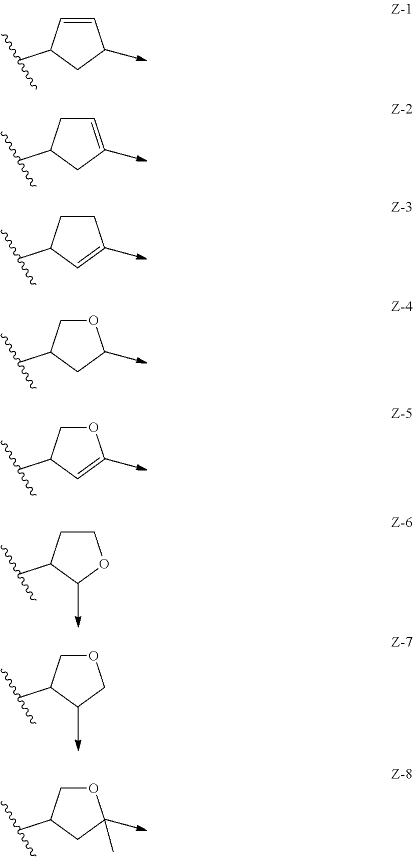
whereas the arrow represents the bonding to the group CO-G of formula (I):
X², X⁴ and X⁶ independently of one another represent hydrogen or fluorine;
X³ and X⁵ independently of one another represent hydrogen, chlorine, cyano or fluorine;
or
represents (C₁-C₃)-Alkyl, (C₁-C₃)-Alkoxy each optionally substituted “m” times by substituents from the group consisting of fluorine or chlorine;
m represents 0, 1, 2, 3, 4 or 5;
n represents 0, 1 or 2;
o represents 0, 1 or 2;
r represents 3, 4, 5 or 6;
or a salt or a combination/composition thereof in a pre-emergence treatment.

10. The method according to claim 5 wherein the crop is a genetically modified plant.
